(19) United States
(12) Patent Application Publication
(10) Pub. No.: US 2009/0259286 A1
(43) Pub. Date: Oct. 15, 2009

(54) Sheath With Radio-Opaque Markers For Identifying Split Propagation

(75) Inventors: Rachit Ohri, Framingham, MA (US); Mark Steckel, Sharon, MA (US)

Correspondence Address: RISSMAN HENDRICKS & OLIVERIO, LLP, 100 Cambridge Street, Suite 2101, BOSTON, MA, 02114, US

(73) Assignee: CAPPELLA, INC., Auburndale, MA (US)

(21) Appl. No.: 12/102,162

(22) Filed: Apr. 14, 2008

(51) Int. Cl. A61F 2/06

(52) U.S. Cl. 623/1.11

(57) ABSTRACT

A medical device delivery system includes a self-expanding medical device mounted on a balloon portion of a catheter. A sheath is provided around the medical device to hold the device in place with the device staying in a compressed state. The balloon portion is inflated to cause the sheath to rupture and release the self-expanding medical device. A number of radio-opaque markers in a pattern that will aid in determining whether or not the sheath has properly ruptured upon inflation of the balloon portion are provided on the sheath. The radio-opaque markers are positioned with respect to an expected sheath rupture propagation path along which the sheath is expected to rupture. The pattern of the markers changes as the sheath ruptures and this change is detected by an operator of the system.

### RELATED APPLICATIONS

### FIELD OF THE INVENTION

The present invention relates to a delivery system for deployment of a medical device, e.g., a self-expanding vascular device such as a stent, in the vasculature of a patient. More particularly, a sheath has radio-opaque markings to aid in the visualization of sheath splitting progress during device delivery.

### BACKGROUND OF THE INVENTION

Treatment of vascular blockages due to any one of a number of conditions can include balloon dilatation and treatment of an inner vessel wall by placement of a tubular prosthesis, e.g., a stent. The stent is positioned to prevent restenosis of the vessel walls after the dilatation. In some instances, a drug eluting stent is used to deliver medicine to the vessel wall to help reduce the occurrence of restenosis.

Stents typically fall into two general categories of construction. The first category of stent is made from a material that is expandable upon application of a controlled force applied by, for example, an inflated balloon portion of a dilatation catheter. The expansion of the balloon causes the compressed stent to expand to a larger diameter that is then left in place within the vessel at the target site. The second category of stent is self-expanding, i.e., formed from shape memory metals or super-elastic nickel-titanium (NiTi or Nitinol) alloys that will automatically expand from a compressed or restrained state when the stent is advanced out of a delivery catheter and into the blood vessel.

Some known delivery systems for implanting self-expanding stents include an inner lumen upon which the compressed or collapsed stent is mounted and an outer restraining sheath that is initially placed over the compressed stent prior to deployment. The outer sheath is moved in relation to the inner lumen to “uncover” the compressed stent, thus allowing the stent to move to its expanded condition. Some delivery systems utilize a “push-pull” type technique in which the outer sheath is retracted while the inner lumen is pushed forward. Still other systems use an actuating wire that is attached to the outer sheath and then pulled to retract the outer sheath and deploy the stent.

There have been, however, problems associated with these delivery systems. For example, systems that rely on a “push-pull” can experience movement of the stent within the body vessel when the inner lumen is pushed forward. This movement can lead to inaccurate positioning and, in some instances, possible perforation of the vessel wall by a protruding end of the stent. Systems that utilize an actuating wire design will tend to move to follow the radius of curvature when placed in curved anatomy of the patient. As the wire is actuated, however, tension in the delivery system can cause the system to straighten. As the system straightens the position of the stent changes because the length of the catheter no longer conforms to the curvature of the anatomy. This change of the geometry of the system within the anatomy can also lead to inaccurate stent positioning.

Delivery systems are known where a self-expanding stent is kept in its compressed state by a sheath positioned about the stent. A balloon portion of the delivery catheter is provided to rupture the sheath and, therefore, release the stent. As shown in U.S. Pat. No. 6,656,213, the stent may be provided around the balloon, with the sheath around the stent, that is, the balloon, stent, and sheath are co-axially positioned, such that expansion of the balloon helps to expand the self-expanding stent as well as rupture the sheath. In other embodiments, the balloon is outside the stent and the sheath surrounds both the balloon and the stent.

There is, however, an issue with respect to the certainty with which it can be determined that the sheath has, indeed, separated sufficiently to release the medical device or stent. It could be dangerous to a patient if the sheath does not rupture sufficiently to release the device. If such a situation occurs, and an attempt is made to withdraw the device and/or delivery system, it is possible that complications could occur—ones that could be fatal. Thus, there is a need to prevent such occurrences.

### SUMMARY OF THE INVENTION

In one embodiment, a sheath for enclosing a medical device on a delivery catheter comprises: a substantially cylindrical tube of material having first and second ends, a longitudinal length, and an outer surface; a rupture initiation portion located near the first end of the tube, the rupture initiation portion defining an expected rupture propagation path; and at least one marker coupled to the tube, the at least one marker positioned with respect to the expected rupture propagation path.

In another embodiment, a medical device delivery system, comprises: a catheter having a distal end and a proximal end; a balloon portion coupled to the catheter; a medical device, having a compressed state and an expanded state, positioned about the balloon portion; and a sheath positioned about the medical device to hold the medical device in the compressed state. The sheath comprises: a substantially cylindrical tube of material having first and second ends, a longitudinal length, and an outer surface; a rupture initiation portion located near the first end of the tube, the rupture initiation portion defining an expected rupture propagation path; and at least one marker coupled to the tube, the at least one marker positioned with respect to the expected rupture propagation path.

In yet another embodiment, a method of manufacturing a sheath for a medical device delivery system comprises: providing a substantially cylindrical tube of material having first and second ends, an outer surface, and a lumen therethrough; creating a rupture initiation portion near the first end of the tube; defining an expected rupture propagation path on the sheath as a function of the location of the rupture initiation portion; and providing at least one marker to the sheath as a function of the rupture initiation portion and the expected rupture propagation path.

### BRIEF DESCRIPTION OF THE DRAWINGS

FIG. 1 is a view of a stent known as an ostial protection device;

FIG. 2A is a view of a known device delivery system;

FIG. 2B is a cross-sectional view of the known device delivery system along line 2B-2B as shown in FIG. 2A;

FIG. 3 is a view of a portion of a known device delivery system;

FIGS. 4 and 5 represent operation of the known delivery system of FIG. 2A in a vessel;

FIG. 6 is a perspective view of a sheath with an initial cut positioned thereon;

FIG. 7 is a perspective view of a sheath in accordance with one embodiment of the present invention;

FIG. 8 is a view of a delivery system incorporating the sheath of FIG. 7;

FIG. 9 is a view of the delivery system of FIG. 8 with partial expansion of a balloon portion and partial rupturing of the sheath;

FIGS. 10A and 10B are methods according to embodiments of the present invention of manufacturing a sheath with markers;

FIGS. 11A-11D portray various sheaths in accordance with various embodiments of the present invention;

FIG. 12 is a cross-section of a sheath representing placement of radio-opaque markers in accordance with various embodiments of the present invention; and

FIGS. 13A-13D portray sheaths in accordance with other embodiments of the present invention.

### DETAILED DESCRIPTION

In known systems, once propagation of the rupturing of the sheath is initiated, the propagation will generally continue without interruption due to the force of balloon expansion and the force due to an emerging end of the self-expanding stent. As will be described below in more detail, embodiments of the present invention provide a mechanism for visualizing and confirming that the sheath has ruptured correctly and/or sufficiently.

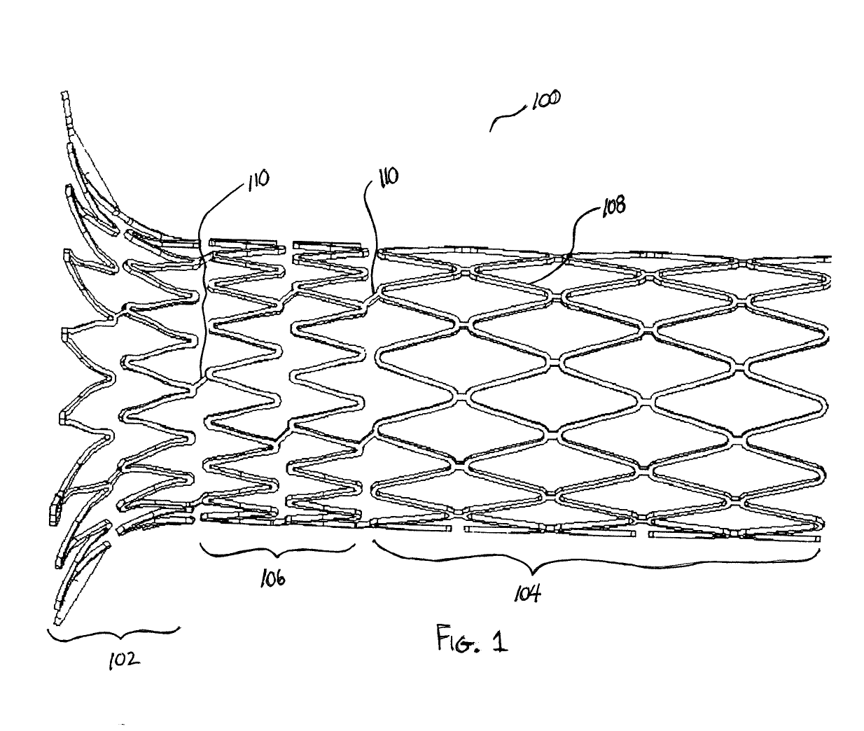

Reference is now made to FIG. 1, which illustrates a schematic view of a medical device 100, in this case, an ostial protection device as described in co-pending U.S. application Ser. No. 11/252,224 filed Oct. 17, 2005, and published as US2007-0061003 on Mar. 15, 2007, for “Segmented Ostial Protection Device,” and which is herein incorporated by reference in its entirety. It should be noted that while the present description refers to an ostial protection device, this is for purposes of explanation only. The claims, unless explicitly recited otherwise, are directed to a sheath for delivering a device into a body vessel and are not limited only to systems with medical devices intended for insertion at an ostium.

The device 100 includes a cap or flared portion 102, an anchor portion 104, and an articulating portion 106. The anchor portion 104 is configured to fit into a side-branch vessel and the cap portion 102 is configured to selectively protect at least part of an ostial region. The articulating portion 106 flexibly connects the anchor portion 104 to the cap portion 102 such that various angles of articulation are possible between each of the three portions. The articulating portion 106 includes connectors 110 connecting to the cap portion 102 and to the anchor portion 104.

The device 100 may be formed of a generally elastic, super-elastic, in-vivo stable and/or “shape-memorizing” material. Such a material is able to be initially formed in a desired shape, e.g., during an initial procedure performed at a relatively high temperature, deformed, e.g., compressed, and then released to assume the desired shape. The device 100 may be formed of Nickel-Titanium alloy (“Nitinol”) that possesses both super-elastic and shape-memorizing properties. Biocompatible non-elastic materials, such as stainless steel, for example, may be also used. The device 100 may be formed from a wire or cut from a single tube of material. The device 100 may be formed from a single piece of material or may be assembled in sections. Other combinations of materials and processes are known and understood by one of ordinary skill in the art.

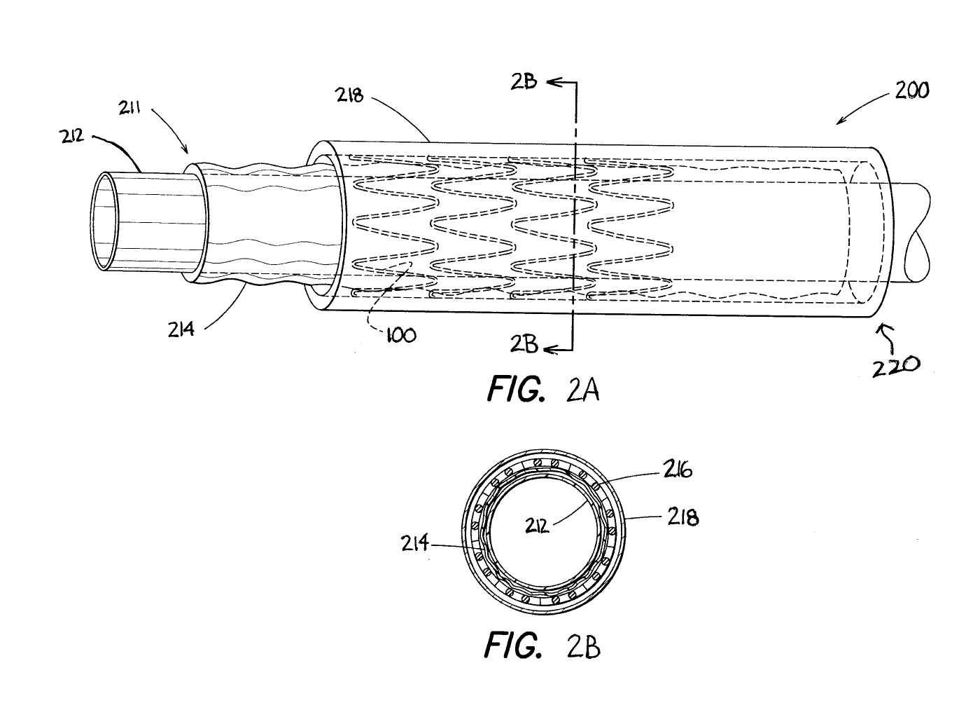

The self-expanding device 100 may be delivered via a system 200, as shown in FIG. 2A, that uses a sheath and a balloon portion of a delivery catheter. One such system is described in US2007-0016280-A1, published Jan. 18, 2007, and entitled “Delivery System And Method Of Use For Deployment Of Self-Expandable Vascular Device,” the entire contents of which is hereby incorporated by reference. In general, as explained in more detail below, the device 100 is compressed and loaded in a low-profile or crimped state about a balloon portion and surrounded by a sheath. To deliver the device, the balloon portion is inflated, causing the sheath to rupture and release the constrained device 100 into its expanded condition within the vessel.

The medical device delivery system 200, as shown in FIG. 2A, includes a delivery catheter 212 with a balloon portion 214 positioned at or near a distal end 211 of the catheter 212. As is known, a lumen is provided to inflate the balloon portion 214 as necessary during the procedure to deliver the device 100 that is placed at or near the distal end of the catheter 212 and around the balloon portion 214. As per the present discussion, the device 100 is a self expanding device and, therefore, a cylindrical sheath 218 is also disposed at or near the distal end 211 of the catheter 212 so as to enclose at least a portion of the device 100 and a part of the balloon portion 214. The sheath 218 is attached to the catheter 212 at some point 220 proximal to the distal end 211 of the catheter 212.

A cross-section view of the system 200, along line 2B-2B, is presented in FIG. 2B. As shown, the sheath 218 surrounds the stent or device 100 and the balloon portion 214 positioned on the catheter 212.

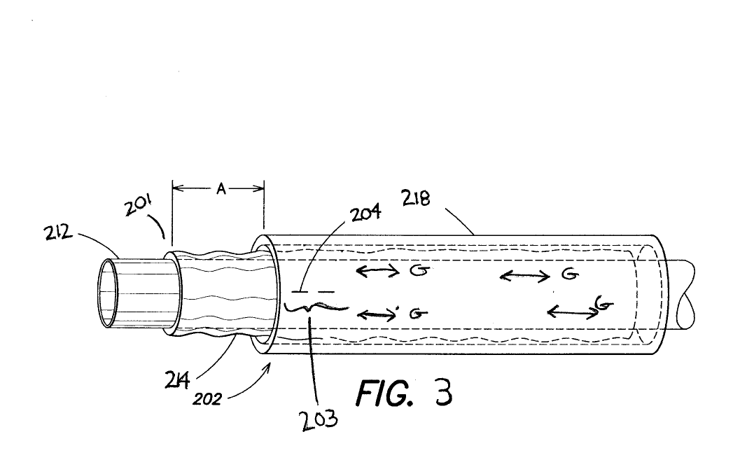

Referring to FIG. 3, a simpler representation (the stent 100 not being shown) of the system 200 of FIG. 2A, is presented where a distal end 202 of the sheath 218 is positioned a distance A proximally from a distal end 201 of the balloon 214. In one embodiment, placing the distal end 202 of the sheath 218 a predetermined distance proximal to the distal end 201 of the balloon 214 allows for maximum effectiveness of the balloon 214 with respect to causing the rupturing of the sheath 218.

In addition, a rupture initiation portion 203 is provided in the sheath 218. In one embodiment, one or more perforations 204 is provided in the sheath 218 as the rupture initiation portion 203. The perforation 204 is shown here near the distal end 202 of the sheath 218. The perforation 204 facilitates separating or rupturing of the sheath 218 as the balloon 214 is expanded. The perforation 204 may comprise, in one embodiment, one or more discontinuous slits, each of a predetermined length, in the sheath material 218. A slit does not necessarily involve the removal of sheath material, as it may comprise a cut from, for example, a sharp edge. Further, in an alternate embodiment, the one or more slits may be of different lengths and the perforations may be of varied size and shape.

Alternatively, the rupture initiation portion 203 may be created by weakening a portion of the sheath 218 by chemical and/or mechanical means with or without penetrating the sheath. Still further, the perforation 204 may comprise one or more holes, where each hole is created by the removal of sheath material. While the perforation 204 is shown here at or near the distal end of the sheath 218, of course, one of ordinary skill in the art would understand that were the sheath 218 to be connected to the catheter 212 at the distal end of the sheath 218, then the rupture initiation portion 203 may be positioned at or near a proximal end of the sheath 218. Further, rupture initiation portion 203 may be provided, in another embodiment, at or near each of the proximal and distal ends of the sheath 218.

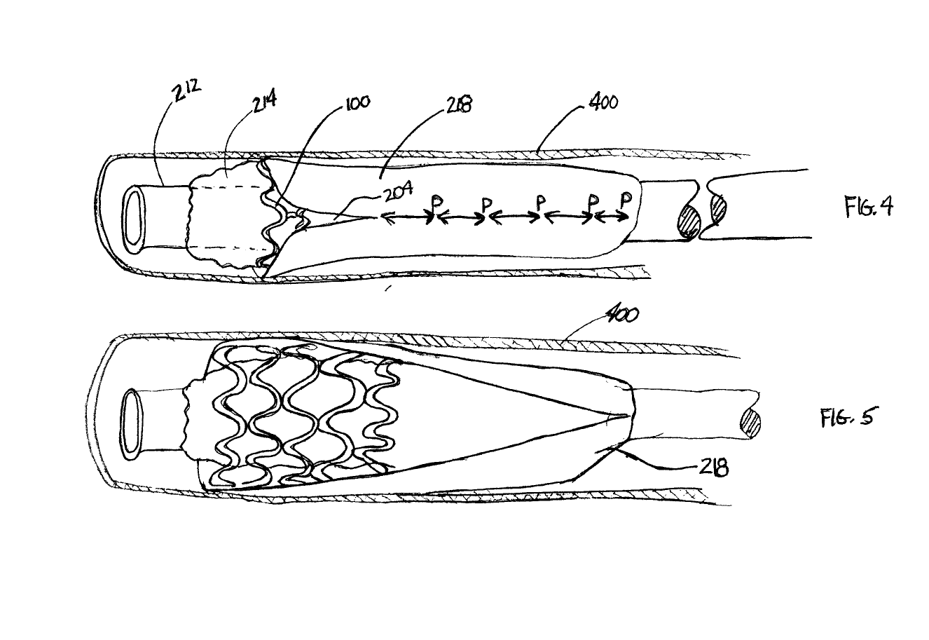

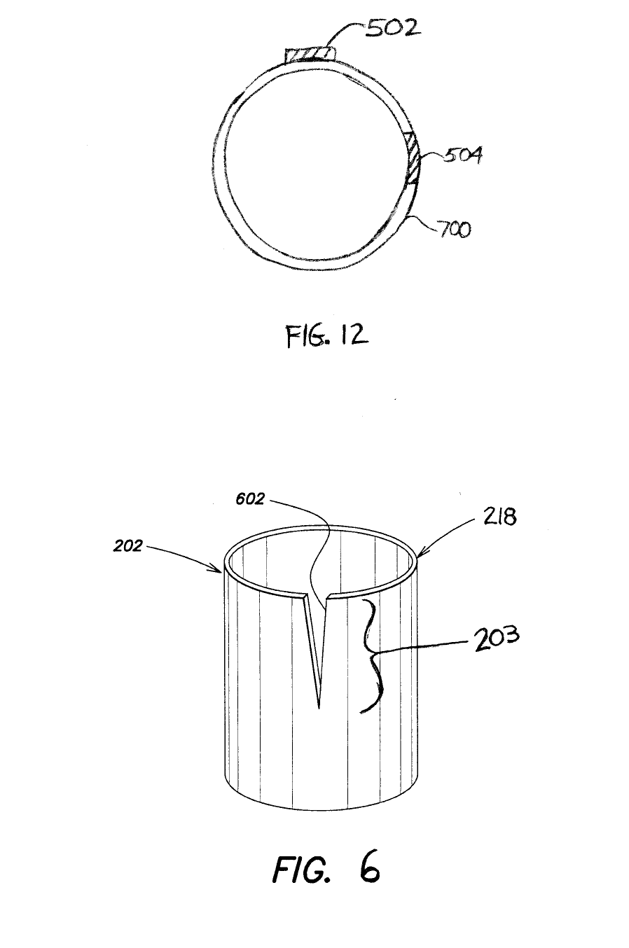

In an another embodiment of the rupture initiation portion 203, a single initial cut 602, as shown in FIG. 6, may be implemented. In the following description, slit and perforation may be used interchangeably to refer to the rupture initiation portion 203.

The sheath 218 may be made from a material such as, for example, PTFE, Nylon, PBAX, and the like. In one embodiment, a sheath made from these types of materials is extruded. The material may take on a characteristic that could be described as having a generally longitudinally-oriented grain. As shown in the figures, the grain G is represented by the arrows. Other materials that may be used for the sheath are understood by one of ordinary skill in the art.

Referring now to FIG. 4, the known delivery system 204, in operation, is positioned at a desired location within a vessel 400. The balloon portion 214 is inflated causing the sheath 218 to rupture. The sheath 218 will start to tear or break along an expected rupture propagation path P as originated or oriented by the initial perforation 204. The expected rupture propagation path P is shown as occurring in a straight line for reasons of clarity in the figures. It should be understood that, in some instances, the expected rupture propagation path P may be a jagged line and not straight. The path P, however, does generally follow a longitudinal direction of the sheath.

Referring to FIG. 5, the sheath 218 will rupture or split as the balloon inflates, and due to the elastic properties of the sheath material, will no longer constrain the device 100. Once the sheath 218 ruptures, the stent expands and is released into the vessel.

It is possible that the rupture propagation is initiated, but not completed, due to anatomical constraints from the vessel, during the deployment of the stent. In such circumstances, it would be beneficial to be able to (a) determine, with some amount of certainty, if the rupture-propagation was initiated, and (b) if initiated, provide an estimate of the propagation progress. Having knowledge on point (a) above will better inform the decision of a physician as to whether or not the stent and delivery system could, or should, be removed from, or repositioned within, the vessel.

Consider the situation or scenario where a sheath does not completely release the device. If the delivery system is withdrawn, the stent may be released in the wrong location. For a device meant to be placed in a side branch vessel, it could result in mis-placement of the device in the main vessel. It is possible that the propagation of the rupture will re-start during withdrawal, even with a deflated balloon, due to the partially emerged distal end of the stent obtaining sufficient room during the withdrawal through the anatomy. In main branch stenting, the result could be a stent that is released at the wrong location.

In accordance with embodiments of the present invention, one or more radio-opaque markers and/or a pattern of radio-opaque markers is provided on the sheath. The position of the markers can be observed during the procedure by, for example, fluoroscopy or other means known to those of ordinary skill in the art. These markers, or patterns of markers, therefore, allow estimation and monitoring of sheath-splitting, i.e., propagation of the rupture, during deployment.

As the balloon-actuated rupture-propagation is initiated during deployment, the resulting change in the radio-opaque pattern of markers, as viewed by an operator or physician, will allow monitoring of the progress of rupture-propagation.

Advantageously, embodiments of the present invention provide a physician with the ability to identify and quantify partial sheath-splitting, e.g., due to anatomical constraints, during a procedure. Such information can be useful during a procedure in order to achieve an optimal outcome.

Referring now to FIG. 7, a sheath 700, in accordance with an embodiment of the present invention, has an initial slit 702 as the rupture initiation portion 203, that extends proximally from a distal end 703 of the sheath 700. The representation of this sheath 700, shown in FIG. 7, is its configuration prior to being placed around a stent and a balloon portion of a delivery catheter and prior to inflation of the balloon. Alternatively, either the rupture initiation portion 203 or the markers may be placed after the sheath is positioned about a device and balloon portion.

A plurality of radio-opaque markers 704 is provided about, or alongside, the expected rupture propagation path P. The placement of the markers 704 is, generally, with respect to the orientation of the initial slit 702 which, in turn, contributes to define the expected propagation path. The slit 702 is generally oriented parallel to a longitudinal axis of the sheath 700 in one embodiment of the present invention.

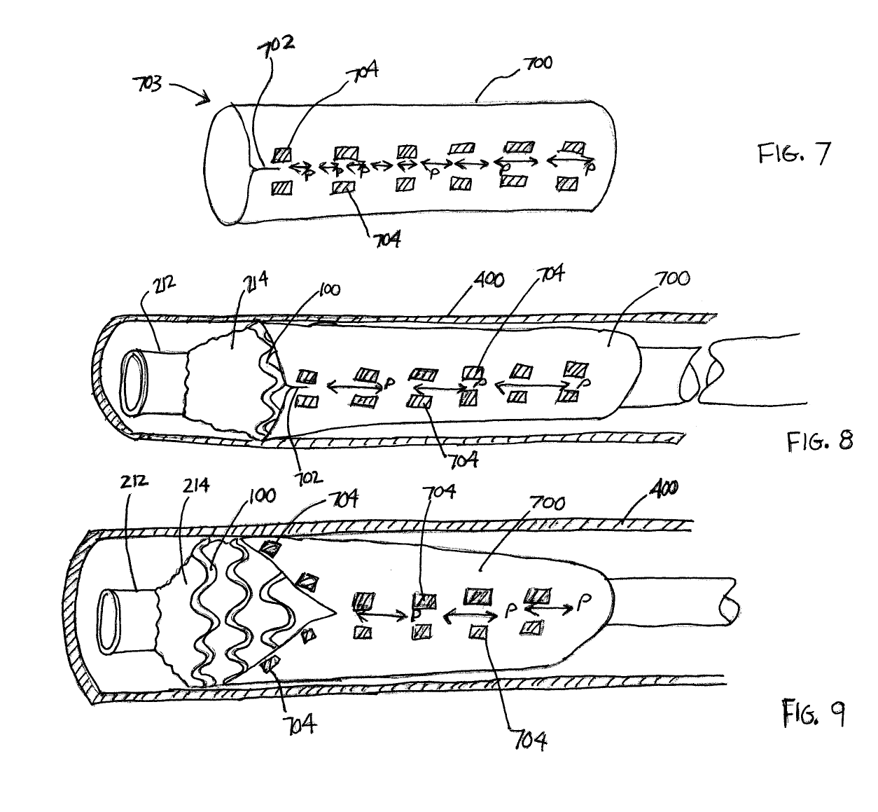

Referring now to FIG. 8, a delivery catheter 212, similar to the one described above is provided with a sheath 700 in place of the known sheath 218. All other elements are similarly referenced or labeled and operate in a manner similar to that which has been described above and as is known to those of ordinary skill in the art.

Upon inflation of the balloon portion 214, as shown in FIG. 9, the pattern of the radio-opaque markers 704 will change. This change in the pattern of the markers 704 will be visible to the physician during the delivery of the stent 100. In operation, the physician will be looking for confirmation that the sheath has split or ruptured sufficiently along its length to confirm that the device has been properly released. If, however, the physician has inflated the balloon and the pattern has not changed, or does not represent proper rupturing, then the physician will know that the device may not have properly deployed. Advantageously, the pattern of the markers 704, when viewed in vivo, may help the physician to analyze the situation and determine what steps need to be taken.

As shown in FIGS. 7-9, the markers are placed “alongside” the expected rupture propagation path P.

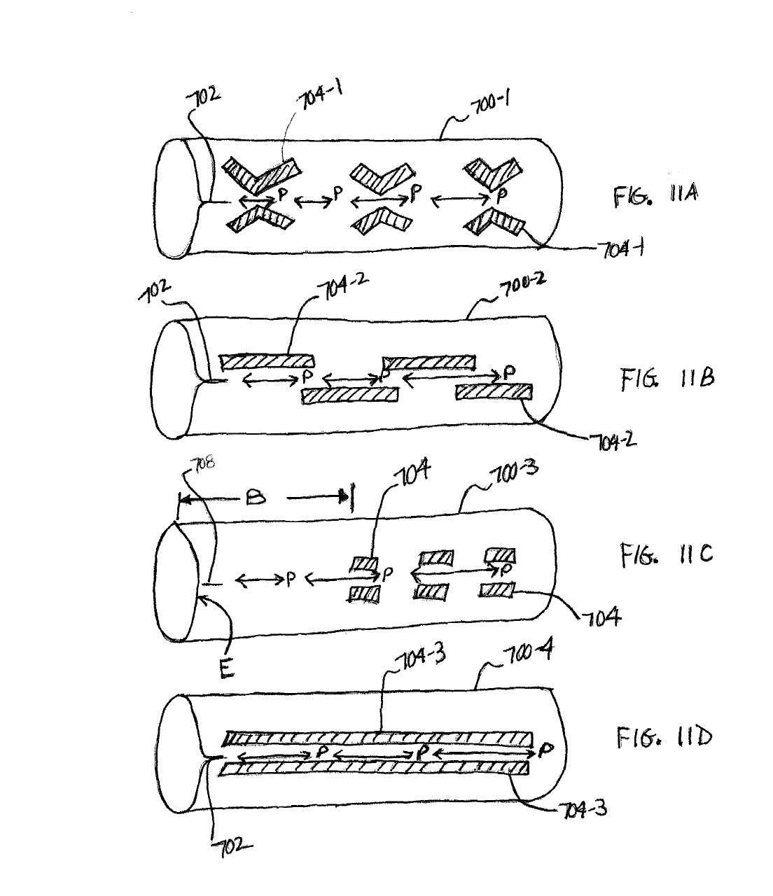

Referring now to FIG. 12, a cross-section of a sheath 700 is presented as an aid to explaining one or more embodiments of the present invention. In one embodiment, a radio-opaque marker 502 is positioned on an outer surface of the sheath. In another embodiment, a radio-opaque marker 504 is embedded, or within, the sheath material. One or more methods for implementing the radio-opaque markers 502, 504 are known to those of ordinary skill in the art.

The radio-opaque markers and/or radio-opaque pattern are placed on the sheath with respect to the expected rupture propagation path P, using, for example, either radio-opaque ink or other radio-opaque materials, such as Gold, Platinum, Iridium, etc. The radio-opaque markers may be implemented with ink imprinting technologies from, for example, Cl Medical, Norton, Mass. One of ordinary skill in the art will understand that other materials may be used to provide the ability to be viewed using fluoroscopy or other similar visualizing approaches in the stenting arts. Further, vapor-deposition and other technologies for depositing these materials are also known to those of ordinary skill in the art.

The size, shape, number, spacing, relative spacing of one marker to another, the material, etc., of the markers 704, are chosen depending upon the particular anatomy in which the device is to be placed. In some circumstances, more markers may be provided than in other circumstances. In one embodiment of the present invention, referring now to FIG. 11A, a sheath 700-1 may comprise markers 704-1 of a shape other than, for example, a square, rectangle, or circle.

The size of each marker is also a function of the chosen radio-opaque material as well as the imaging technology used in conjunction with the procedure. The size of the marker must not be so small that it cannot be detected, i.e., it should not be smaller than a pixel-size, or resolution capability, of the detecting apparatus or system.

In another embodiment of the present invention, a sheath 700-2 comprises markers 704-2 that are longitudinally offset from one another, as shown in FIG. 11 B. This relative orientation of the markers to one another may also provide an advantageous visual aid in some circumstances. Of course, the longitudinal opposition or offset of markers can be combined with different combinations of marker shapes and the drawings are not intended to be limiting but only to serve as examples for explanatory purposes.

In one embodiment of the present invention shown in FIG. 11 C, a sheath 700-3 includes a slit 708 that does not extend all of the way to an edge or distal end E of the sheath. The location of the slit 708 “set-back” from the edge E will still allow for proper sheath rupture. In addition, with the slit 708 set-back from the distal end E, the rupture may proceed in two directions, i.e., from the slit 708 along the expected path toward the opposite, proximal, end and also toward the edge E. As shown in FIG. 11C, markers 704 are placed no closer than a predetermined distance B from the distal end E at which the slit 708 is placed. This may be implemented in the situation where it is expected that the sheath will readily split at those locations closer to the rupture initiation portion 203 or initial slit 708, but those locations farther from the initial slit 708 are the ones that need to be observed.

In yet another embodiment of the present invention as shown in FIG. 11D, a sheath 700-4 comprises a pair of markers 704-3 that runs substantially the full length of the expected rupture propagation. In this embodiment, the markers 704-3 function as a “guide” between which the rupture will travel.

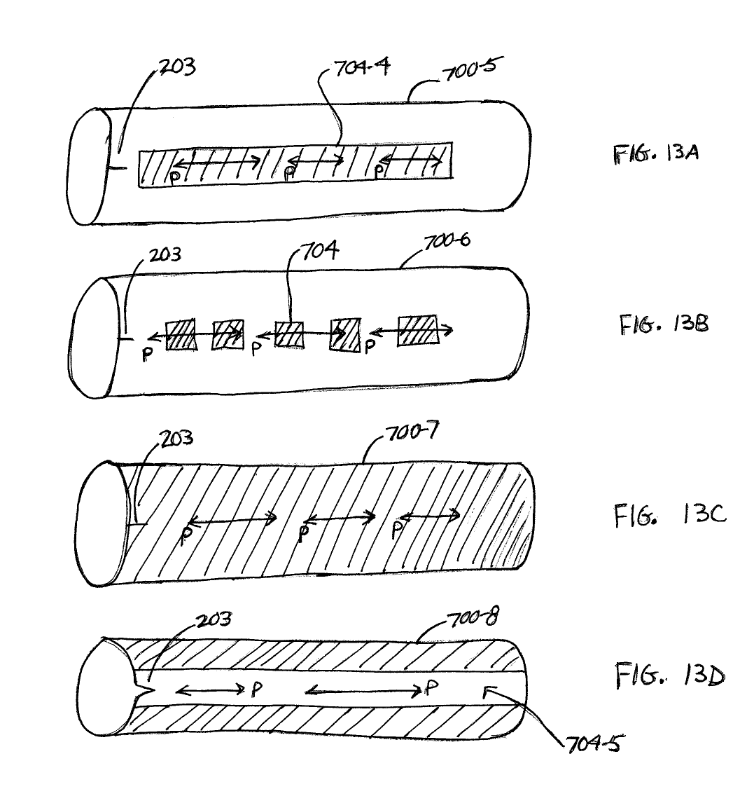

The embodiments of the present invention described above are directed to providing radio-opaque markers that are positioned alongside, i.e., on each side of, the expected propagation path P. Referring now to FIGS. 13A-13C, other embodiments of the present invention provide radio-opaque markers that “straddle” or cross the expected rupture propagation path P.

A sheath 700-5, as shown in FIG. 13A, comprises an initiation portion 203, which could be a slit or perforation as described above, and a radio-opaque stripe 704-4. As shown, the stripe 704-4 is positioned such that the expected propagation path P runs through it. As the balloon portion is inflated, and the sheath 700-5 ruptures, the radio-opaque stripe 704-4 will split into two pieces. The splitting of the stripe 704-4 can be observed by the physician to confirm the progress of the release of the device.

Alternatively, a sheath 700-6, in accordance with another embodiment of the present invention, is provided with a plurality of radio-opaque markers 704 provided so as to straddle the expected rupture propagation path P as shown in FIG. 13B. Of course, the markers 704 can be placed astride the expected path P in conjunction with the embodiments described above with respect to, for example, any choices as to the setback from the distal end, the size, or the shapes of the markers.

In another embodiment, the sheath 700-6 includes markers 704 chosen so as to be large enough to be visible by the imaging system when whole, i.e., prior to balloon rupture, but too small to be seen once the rupture has “broken” the marker 704. Accordingly, the markers 704 will seem to disappear from view as the sheath is rupturing.

Further, referring to FIG. 1 3C, a sheath 700-7, in accordance with yet another embodiment of the present invention, is made from material that is entirely radio-opaque. Thus, the expected propagation path P will alter the image seen by the physician as the sheath is being ruptured. Accordingly, the progress of the deployment of the device can be monitored.

Still further, referring to FIG. 13D, a sheath 700-8, in accordance with yet another embodiment of the present invention, is made from material that is radio-opaque except for a stripe 704-5 that is astride the expected rupture propagation path P but is not radio-opaque.

The sheaths described in FIGS. 13A-13D may be implemented by one or more of the methods as listed above in addition to those known to those of ordinary skill in the art. Further, the radio-opaque markers may be provided on or within the sheath by operation of a process where the radio-opaque material and the sheath material are co-extruded, or by vapor deposition.

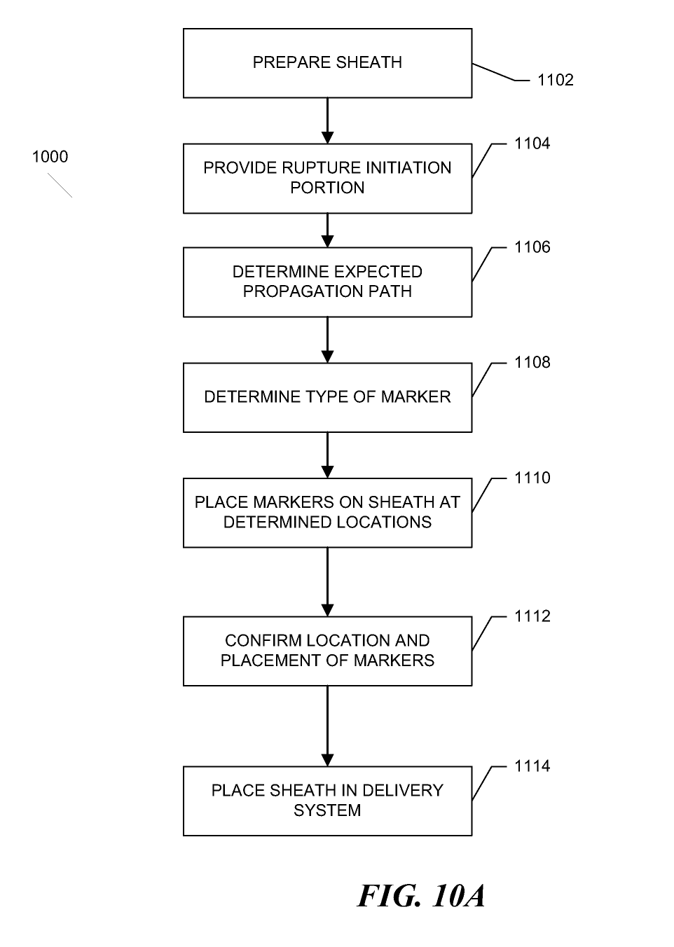

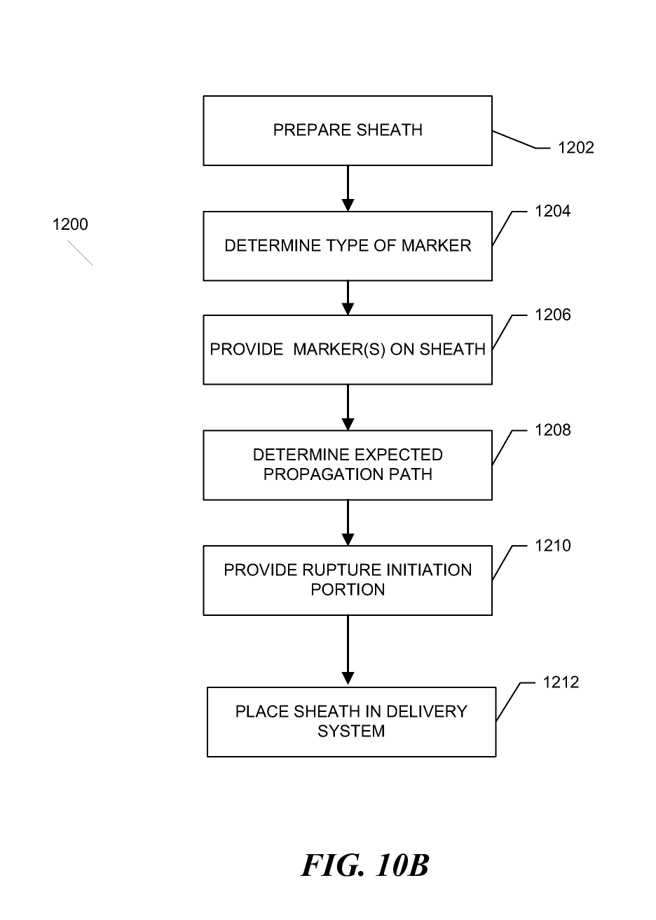

One embodiment of the present invention is a method 1000 for making a sheath with propagation markers, referring now to FIG. 10A. Initially, step 1102, the sheath is prepared from an appropriate material. Subsequently, step 1104, the rupture initiation portion is provided in the sheath as either an initiation slit or one or more perforations. Once the rupture initiation portion is provided in the sheath, then the expected rupture propagation path P can be determined, step 1106. This step may involve providing some visual aids to indicate where the expected rupture propagation path is located, for example, temporary ink on the sheath itself or markings on a support mandrel supporting the sheath. The type of marker, for example, the material, the shape of the markers, the number of markers, the locations of the markers with respect to the expected rupture propagation path, as well as other attributes, are determined at step 1108. Once the characteristics of the markers are determined, at step 1110, the markers are provided in or on the sheath at the determined locations. As part of a quality control process, step 1112, the locations and proper placement of the markers can be confirmed. Once the sheath is accepted, it can be placed in a delivery system, step 1114.

The foregoing method of making a sheath in accordance with one embodiment of the present invention is not limited to the specific steps and the order set forth above. One of ordinary skill in the art will understand that the steps and/or the order of the steps can be altered, for example, depending upon the technologies used to provide the radio-opaque material on the sheath. That is, the method may differ if an extrusion process instead of a “deposition” process is used.

Thus, a method 1200 of making a sheath in accordance with yet another embodiment of the present invention is set forth in FIG. 10B. Initially, step 1202, the sheath is prepared from an appropriate material. The type of marker, for example, the material, the shape of the markers, the number of markers, the relative locations of the markers, etc., are determined at step 1204. Once the characteristics of the markers are determined, at step 1206, the markers are provided in or on the sheath at the determined locations. The markers may be provided by deposition or co-extrusion or any other method known to one of ordinary skill in the art. Once the markers are provided, then the expected rupture propagation path P can be determined, step 1208. Subsequently, step 1210, the rupture initiation portion is provided in the sheath. As above, in one embodiment of the present invention, an initiation slit is provided at or near a distal end of the sheath and is oriented parallel to the longitudinal length of the sheath. Once the sheath is accepted by any quality control checks, it can be placed in a delivery system, step 1212.

It is to be understood that the embodiments of the present invention are not limited in their application to the details of construction and the arrangement of the components set forth in the foregoing description or illustrated in the drawings. Also, it is to be understood that the phraseology and terminology employed herein are for the purpose of description and should not be regarded as limiting.

It is further appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although various exemplary embodiments of the present invention have been disclosed, it will be apparent to those skilled in the art that changes and modifications can be made that will achieve some of the advantages of the invention without departing from the spirit and scope of the invention. It will be apparent to those reasonably skilled in the art that other components performing the same functions may be suitably substituted.

What is claimed is:

1. A sheath for enclosing a medical device on a delivery catheter, the sheath comprising:
a substantially cylindrical tube of material having first and second ends, a longitudinal length, and an outer surface;
a rupture initiation portion located near the first end of the tube, the rupture initiation portion defining an expected rupture propagation path; and
at least one marker coupled to the tube, the at least one marker positioned with respect to the expected rupture propagation path.

2. The sheath of claim 1, wherein the at least one marker is at least one of:
disposed on the outer surface of the tube material; and
disposed in the outer surface of the tube material.

3. The sheath of claim 2, wherein:
the at least one marker comprises radio-opaque material.

4. The sheath of claim 3, wherein the rupture initiation portion comprises at least one of:
a slit running for a predetermined length from the first end of the tube toward the second end of the tube;
at least one perforation; and
a weakened portion of the sheath material.

5. The sheath of claim 1, wherein the at least one marker comprises:
a first pair of markers, and
wherein the expected rupture propagation path passes between the markers in the first pair.

6. The sheath of claim 1, wherein no markers are positioned within a predetermined distance of the first end of the tube.

7. The sheath of claim 1, wherein the at least one marker is located such that the expected rupture propagation path passes through it.

8. The sheath of claim 7, further comprising:
a plurality of markers disposed in a pattern such that the expected rupture propagation path passes through each marker.

9. A medical device delivery system, comprising:
a catheter having a distal end and a proximal end;
a balloon portion coupled to the catheter;
a medical device, having a compressed state and an expanded state, positioned about the balloon portion; and
a sheath positioned about the medical device to hold the medical device in the compressed state, the sheath comprising:
a substantially cylindrical tube of material having first and second ends, a longitudinal length, and an outer surface;
a rupture initiation portion located near the first end of the tube, the rupture initiation portion defining an expected rupture propagation path; and
at least one marker coupled to the tube, the at least one marker positioned with respect to the expected rupture propagation path.

10. The delivery system of claim 9, wherein:
the at least one marker comprises radio-opaque material.

11. The delivery system of claim 9, wherein the rupture initiation portion comprises at least one of:
a slit running for a predetermined distance from the first end of the tube toward the second end of the tube;
at least one perforation; and
a weakened portion of the sheath material.

12. The delivery system of claim 9, wherein the at least one marker comprises:
a first pair of markers,
wherein the expected rupture propagation path is disposed between the markers in the first pair.

13. The delivery system of claim 9, wherein no markers are positioned within a predetermined distance of the first end of the tube.

14. The delivery system of claim 9, wherein the at least one marker is located such that the expected rupture propagation path passes through it.

15. A method of manufacturing a sheath for a medical device delivery system, the method comprising:
providing a substantially cylindrical tube of material having first and second ends, an outer surface, and a lumen therethrough;
creating a rupture initiation portion near the first end of the tube;
defining an expected rupture propagation path on the sheath as a function of the location of the rupture initiation portion; and
providing at least one marker to the sheath as a function of the rupture initiation portion and the expected rupture propagation path.

16. The method of claim 15, further comprising:
providing the at least one marker with radio-opaque material.

17. The method of claim 15, wherein providing the at least one marker comprises:
providing a first pair of markers such that the expected propagation path is between them.

18. The method of claim 15, further comprising:
locating the at least one marker such that no markers are within a predetermined distance of the first end of the tube.

19. The method of claim 15, wherein creating the rupture initiation portion comprises at least one of:
creating a slit in the tube running for a predetermined distance from the first end of the tube toward the second end of the tube;
weakening a portion of the sheath material; and
punching at least one hole in the sheath material.

20. The method of claim 15, further comprising:
locating the at least one marker such that the expected rupture propagation path passes through it.
